(19) United States
(12) Patent Application Publication
(10) Pub. No.: US 2012/0065115 A1
(43) Pub. Date: Mar. 15, 2012

(54) CARA B Naturally - Shampoo / Body Wash

(76) Inventor: Landra Booker Johnson, Charlotte, NC (US)

(21) Appl. No.: 12/826,433

(22) Filed: Sep. 12, 2010

(51) Int. Cl. A61K 8/97; A61Q 19/10; A61Q 5/02

(52) U.S. Cl. 510/121; 510/130

(57) ABSTRACT

The hair shampoo/body wash composition of the present invention utilizes all natural plant derived components as a method for cleansing, conditioning and moisturizing the hair. The invention is a “tear-free” type composition and provides good moisturizing and gives a smooth, soft touch feel upon using on the skin and hair, and is excellent in stability.

Product: Shampoo/Body Wash

| ID# | Phase | Ingredients | % by WT |
|---|---|---|---|
| N/A | A/E | Water | 59.6863 |
| 591409 | C | Ritafactant 138AN | 35.0000 |
| 591407 | B | Guar Hydroxypropyltrimonium Chloride | 0.5000 |
| 590095 | A | Vegetable Glycerin 99.7% USP | 1.0000 |
| 590055 | H | Biovert Substrate (Glucose) | 1.0000 |
| 590054 | H | Biovert Enzyme | 0.0500 |
| | | (Glucose Oxidase/lactoperoxidase) | |
| 571738 | G | Citric Acid | 0.5000 |
| 591797 | F | Milk & Chamomile #74962 | 2.0000 |
| 591541 | E | Carmael AP100 | 0.0637 |
| 591607 | F | Tocopheryl Acetate | 0.1000 |
| 590284 | F | Aloe Barbadensis Leaf Juice | 0.1000 |
| | | TOTAL: | 100.0000 |

I claim:

1. A one-part all natural hair shampoo and body wash composition comprising the following ingredients (A) to (K): (A) from greater than 50% deionized water; (B) 25 to 50% ritafactant; (C) 1 to 5% natural fragrance #74962; (D) 1 to 5% vegetable glycerin;
(E) 1 to 5% biovert substrate; (F) 0.1 to 1% tocopheryl acetate; (G) 0.1 to 1% aloe vera barbadensis leaf juice; (H) 0.1 to 1% citric acid; (I) 0.1 to 1% guar hydroxypropytrimonium chloride; (J) less than 0.1% natural caramel color; and (K) less than 0.1% biovert enzyme.
